(19) United States
(12) Patent Application Publication
(10) Pub. No.: US 2010/0240977 A1
(43) Pub. Date: Sep. 23, 2010

(54) METHOD FOR MEASURING THE RESPONSE OF A TISSUE TO AN ELECTROMAGNETIC FIELD

(76) Inventor: Andreas Caduff, Zurich (CH)

Correspondence Address: LADAS & PARRY LLP, 26 WEST 61ST STREET, NEW YORK, NY, 10023, US

(21) Appl. No.: 12/602,909

(22) PCT Filed: Jun. 20, 2007
(86) PCT No.: PCT/CH07/00305

§ 371 Date: Apr. 27, 2010

(51) Int. Cl. A61B 5/145; A61K 49/00

(52) U.S. Cl. 600/365; 424/9.1

(57) ABSTRACT

The present invention relates to a method for measuring the response of the tissue of a human or animal body to an electromagnetic field, comprising the steps of i) treatment of a skin area of said body with a topical composition comprising one or more reversible mnAChR-antagonists, ii) applying to the treated skin area a measuring device and iii) measuring by means of said measuring device at least one parameter depending on a response to said electromagnetic field; to a method for measuring in vivo the glucose content in blood and to topical compositions useful in said methods

The present invention relates to a method for measuring the response of the tissue of a human or animal body to an electromagnetic field; to a method for measuring in vivo the glucose level in the body and in particular in blood and to topical compositions useful in said methods.

The determination of physiological parameters of the human or animal body is relevant, for example to establish a diagnosis or to appropriately treat a disease or malfunction of said body. In this context, it is advantageous to establish non-invasive methods. Further, in specific applications it is advantageous to use methods that provide measuring results continuously. Various devices that provide such measuring results, such as glucose monitors or cardiac function monitors must be worn on the skin. The measuring results obtained by such devices are deficient e.g. due to i) variability of the skin, ii) moisture loss and iii) possible hypersensitivity.

A parameter of particular relevance is the glucose content of blood. The in vitro determination of glucose is known in the field.

Methods of in vivo determination of glucose are described, e.g. in WO02/069791; WO2005/053526; WO2005/053523 the content of these documents is incorporated by reference. These methods provide good results; however, they suffer from long measuring times and/or limited reliability.

Further, WO04/093833 discloses a two-sided pad, where the first side contains a cleansing formulation and the second side contains a hydrating formulation, for enhancing the performance of transdermal sensing, delivery or monitoring devices.

Thus, it is an aim of the present invention to provide an improved method for the in vivo determination of physiological parameters which overcomes the disadvantages of the known methods.

This is achieved by a method as described in claim 1. Further aspects of the invention are disclosed in the independent claims and the specification. Advantageous embodiments are disclosed in the dependent claims and the specification.

The present invention will be described in more detail below. It is understood that the various embodiments, preferences and ranges as provided/disclosed in this specification may be combined at will. Further, depending of the specific embodiment, selected definitions, embodiments or ranges may not apply.

In the context of the present invention, the following definitions shall apply.

The term “electromagnetic field” is known; it e.g. encompasses high frequency fields (e.g. 1 MHz to 10 GHz), low frequencies fields below 1 MHz (e.g. 60 Hz), UV-, visible and infrared light (0.2-100 μm) as well as microwave radiation. Such fields are generated by one or more field generating electrodes (low/high frequencies field), by one or more light sources emitting light in the range of 0.2-100 μm or by one or more microwave generators.

The term “measuring device” denotes any device capable of measuring at least one parameter of the tissue that is responsive to an electromagnetic field. Typically (but not necessarily), the measuring device also generates the electromagnetic field.

The term “topical formulation” defines any formulation that can be applied to the skin. Such formulations are known in the field and comprise (sprayable) liquids, crèmes, gels.

Compounds that act as reversible antagonists of the muscular nicotinic acetylcholine receptor (“mnAChR antagonists”) are known and/or may be identified by standard tests. Preferred are those mnAChR antagonists that act at the post synaptic membrane. Examples of such compounds include natural and synthetic tripeptides such as a compound of formula (I)

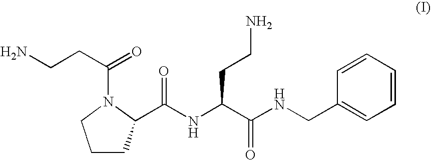

and its salts, e.g. the diacetate salt.

In a first aspect, the invention relates to a method for measuring the response of a tissue of a human or animal body to an electromagnetic field comprising the steps of i) treatment of a skin area of said body with a topical composition comprising one or more reversible mnAChR-antagonists, ii) applying to the treated skin area a measuring device and iii) measuring by means of said measuring device at least one parameter depending on a response to said electromagnetic field. The tissue of a human or animal body refers to any tissue of said body; preference is given to the skin and the underlying tissues. As is generally appreciated, skin is composed of a number of tissues, in particular dermis and epidermis, forming the organ “skin”. Thus, also the response of a number of tissues is encompassed by the disclosed method. For avoidance of doubt, blood is also considered a tissue in the context of the present invention. A response of a tissue refers to the fact that an applied electromagnetic field leads to physical processes in the tissue which can be measured. For example, the applied electromagnetic field may be absorbed, scattered, reflected or phase shifted at least in part or a current may be induced, which in turn can be measured. The treatment of a skin area refers to the application of an effective amount of a topical composition comprising one or more reversible mnAChR-antagonists to the skin. A suitable skin area has advantageously a size of 1-100 cm². It may also be possible to treat more than one area for the method as described herein. For example, the areas where the electromagnetic field is applied and the area where the response is measured may be treated. Alternatively, only the area where the response is measured is treated. Such treatment may be repeated, as the case may be. For example, skin is treated once a day. To apply a measuring device refers to a step that enables the measuring device to detect the response of the skin. Said device may be in direct contact with the skin or it may be separated from direct skin contact, e.g. by a positioning an inert material between skin and measuring device.

In a preferred embodiment, the electromagnetic field used in the method as described herein is applied by means of one or more electrodes (“field generating electrodes”). These electrodes may generate an electromagnetic field of 1 kHz to 10 GHz. Such electromagnetic field will cause a response of the tissue, e.g. by changing the electromagnetic field, which is measured by the electrodes of the measuring device (“measuring electrodes”). In case an electromagnetic field is applied, the measuring device will contain one or more electrodes capable of measuring said response. Field generating electrodes and measuring electrodes may be identical or separate electrodes. Advantageously, said one or more electrodes are applied to the treated skin area as described above. The step of “applying an electrode to the treated skin area” refers to an arrangement of electrode(s) and treated skin area in a way that allows generating an electromagnetic field and measuring the response of the tissue to the applied electromagnetic field. The electrodes of the measuring device may be in the same area as the electrodes of the field generating device or in any other suitable position. Thus, the electrodes referred to above may be located directly on the skin or may be separated from direct skin contact, e.g. by a positioning of an inert material between skin and electrode.

In a further preferred embodiment, the electromagnetic field used in the method as described herein is applied by means of one or more light sources emitting UV-, visible or infrared light. Preference is given to light of a wavelength between 0.8-100 μm. In this spectral range, water gives rise to strong optical absorption, for which reason the application of the present invention is particularly advantageous. Preference is given to a method wherein said light is incident to said treated skin area. A suitable measuring device in the context of this embodiment comprises one or more light-sensitive sensors. Such sensors may be arranged at the same or at an opposite side of the emitting light source.

In a further advantageous embodiment, the reversible mnAChR-antagonist used in the method as described herein is selected from the group consisting of natural and synthetic tripeptides; in particular Compound of formula (I). The term tripeptides relates to a peptide consisting of three amino acids joined by peptide-moieties [—N(R)—C(O)—; wherein R represents hydrogen or (cyclo)-alkyl]. Said amino acids may be natural or synthetic. Those tripeptides occurring in nature are regarded as “natural tripeptides”, in contrast to “synthetic tripeptides”. Depending on the formulation used in the context of this invention, tripeptides or their pharmaceutically acceptable salts (e.g. the acetate salt) may be employed. The tripeptides may posses one or more chiral carbon atoms; thus enantiomers and diastereomers of tripeptides may exist. Within the scope of this invention, all possible optical isomers are encompassed. Preference, however, is given to those tripeptides having the S-configuration on all asymmetric carbon atoms.

In a further advantageous embodiment, the topical composition as described above is formulated as a liquid, a gel or a cream. Suitable excipients for manufacturing a gel, cream or liquid are known in the field. The term liquid formulation includes solutions and suspensions.

In a further advantageous embodiment, the human body is subject to a method as described herein.

In a further advantageous embodiment, the invention relates to a method for measuring in vivo the response of one or more tissue(s), in particular blood, to an electromagnetic field.

In a second aspect, the invention relates to a method for measuring in vivo the glucose content in blood, comprising the step i) treatment of a skin area of said body with a topical composition comprising one or more reversible mnAChR-antagonists, ii) applying to the treated skin area a measuring device and iii) measuring by means of said measuring device at least one parameter depending on a response to said electromagnetic field and iv) determine the glucose-content. Steps i), ii) and iii) are already described above in the context of a method for measuring the response of a tissue of a human or animal body to an electromagnetic field (“first aspect”). Thus, the various preferences and advantageous embodiments may be applied to the second aspect likewise. Step iv), the determination of the glucose content, may be achieved by comparing the measuring results with a calibration curve.

In a third aspect, the invention relates to a topical composition, comprising 10⁻¹ to 10⁻³ wt-%, preferably 0.005-0.02 wt-%, of an reversible mnAChR antagonist, such as a compound of formula (I). It was surprisingly found that such topical compositions may be used in a method as described above. The use of such topical composition remarkably improves a measuring method involving the response of a tissue of a human or animal body to an electromagnetic field. Thus, results are obtained faster and more reliable. Without being bound to theory, it is believed that a reversible mnAChR antagonist, such as a compound of formula (I), when applied in an effective concentration, causes a down regulation of perspiratory glands and thus improves the response of a tissue to an electromagnetic field. It was found by the inventors that a suitable topical composition to be used in the context of the invention will address the following issues: i) at least down regulation of perspiratory glands; ii) optionally control of transepidermal water flow (TEWL); iii) optionally moisturizing of skin. These objectives are met by a topical composition as described herein.

A topical composition according to this invention may contain further components such as active ingredients from the class of occlusive agents, humectants, emollients and known excipients suitable for topical compositions. The following provides an overview on suitable further components.

Occlusive agents are hydrophobic agents capable of forming an occlusive film on the skin to reduce TEWL by preventing evaporation of water from the S.C. Occlusive agents may also help to restore the lipid barrier of the skin. Occlusive agents are known in the field and include lipids and petrolatum. Lipids may assist in retaining the moisture content of the skin or particular tissues of the skin, while petrolatum may reduce TEWL.

Humectants are agents that attract water to the skin. Typically, the water is drawn from the deeper dermis, rarely the environment. Humectants are known in the field and include glycerol, pahtehnol, ammonium lactate, hyaluronic acid, alpha-hydroxy amino acids (in particular lactic acid and its sodium salt, glycolic acid, malic acid, tartaric acid alone or in combination), the group of natural moisturizing factors (“NMFs”; in particular urea, pyrrolidoncarboxylic acids, lactate, citrate, formiate, phosphate, amino acids, ammonia, uric acid alone or in combination) It was surprisingly found that the addition of a certain amount of natural moisturizing factors (NMFs), such as urea, pyrrolidoncarboxylic acids, lactate, citrate, formiate, phosphate, aminoacids, ammonia, uric acid alone or in combination, improves the properties of the topical composition as described above.

Emollients are substances having a softening or smoothing effect to the skin or the mucous membrane. Emollients are known in the field and include silicones, triglycerides (in particular essential fatty acids, waxes and ceraceous substances, fatty alcohols, sterol), saturated and unsaturated hydrocarbons (in particular paraffin and Petrolatum)

Excipients are inactive substances used as a carrier, filler and the like for a topical composition as described above. Suitable excipients are known in the field and can be chosen by a person skilled in the art.

Preferred additional components will be described below:

It was surprisingly found that the addition of a certain amount of urea further improves this effect. This may be associated to an interaction between water and urea which stabilizes the water content of the skin tissue. In fact, a reversible mnAChR antagonist, e.g. a compound of formula (I), and urea positively influence each other, providing a synergistic effect. Thus, in a preferred embodiment, the invention also relates to a topical composition comprising 10⁻¹-10⁻³ wt-% of a reversible mnAChR antagonist, e.g. a compound of formula (I), and an effective amount of urea, e.g. 0.1-10 wt-% urea.

It was also surprisingly found that the addition of a certain amount of hyaluronic acid further improves this effect. This effect may be associated to water-binding properties of hyaluronic acid. Although it is speculated that hyaluronic acid does not penetrate through the skin, it decreases TEWL and increases the hydration of the skin. In fact, a reversible mnAChR antagonist, e.g. a compound of formula (I), and hyaluronic acid positively influence each other, providing a synergistic effect. Thus, in a preferred embodiment, the invention also relates to a topical composition comprising 10⁻¹-10⁻³ wt-% of a reversible mnAChR antagonist, e.g. a compound of formula (I), and an effective amount of hyaluronic acid, e.g. 0.01-10 wt-%, preferably 0.01-1 wt-% hyaluronic acid.

It was also surprisingly found that the addition of a certain amount of glycerol further improves this effect. In fact, a reversible mnAChR antagonist, e.g. a compound of formula (I), and glycerol positively influence each other, providing a synergistic effect. Thus, in a preferred embodiment, the invention also relates to a topical composition comprising 10⁻¹ to 10⁻³ wt-% of a reversible mnAChR antagonist, e.g. a compound of formula (I), and an effective amount of glycerol, e.g. below 30 wt-%, preferably below 20 wt-%, such as 2-3 wt-% glycerol.

The invention also relates in a further embodiment to a topical composition as described herein formulated as a pharmaceutical solution, gel or cream. The viscosity may vary in a broad range from almost solid (comparable to a lipstick) to thin fluid (comparable to a sprayable solution). Such formulations are suitable for topical applications. Gels and creams are considered advantageous due to a low allergic potential, good absorption and a high hydrating effect.

The invention also relates in a further embodiment to the manufacture of a topical composition as described herein. Such formulations may be manufactured according to known procedures using known excipients, e.g. the components as described above. Thus, a topical formulation as described herein is obtainable by preparing an o/w or w/o emulsion according to known procedures first (e.g. combining all lipophilic components and all hydrophilic components separately, advantageously at temperatures of 60-80° C., than adding the hydropohilic phase to the lipophilic phase while stirring) than one or more mnAChR antagonist, optionally diluted in a suitable diluent (e.g. glykole/water) and optionally further components are added to the above described emulsion, advantageously at a temperature between 0° C. and 45° C., e.g. 35° C. Further details are provided in the examples.

The topical formulation as described herein may be supplied to the consumer in standard packages, like tube or dispenser. However, it was found advantageous to provide specifically adapted application devices to obtain best results for the measuring method as described above. Thus, the invention also relates in a further embodiment to an application device specifically adapted to the application of a topical composition as described herein. Such application devices include single sachets and blister like packages. In an advantageous embodiment, the application device contains two compartments, one containing the topical composition as described herein, the other one a cleansing composition. Such devices are considered advantageous, as a predefined amount of formulation is applied to the skin. The compartments are thus advantageously adapted to host a volume of 0.5-5 mL each.

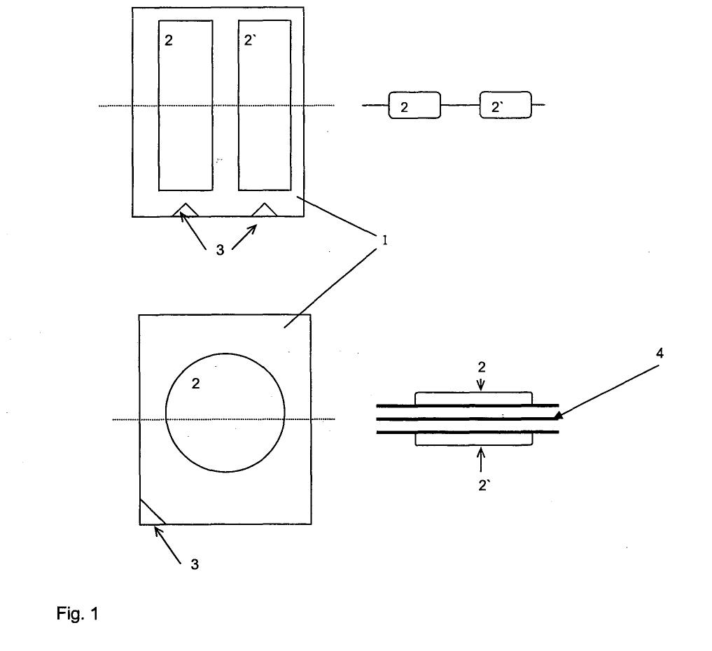

In one embodiment, the compartments are arranged side-by side, each compartment having a means for opening. FIG. 1a shows an example of such an application device (1) with compartments for cleansing formulation and topical formulation (2, 2′) and opening device (3).

In an alternative embodiment, the two compartments are arranged face-to-face, each compartment having a means for opening. FIG. 1b shows an example of such an application device (1) with compartments for cleansing formulation and topical formulation (2, 2′) separated by a central sheet (4) and opening device (3).

Suitable cleansing compositions are any compositions known in the field for cleaning the skin. Typically, such formulations contain one or more alcohols, such as ethanol or iso-propanol, optionally water and optionally perfumes. Typically, such compositions are formulated as a liquid.

In a fourth aspect, the invention relates to the use of a topical composition as described herein in a method for measuring the response of a tissue of a human or animal body to an electromagnetic field or in a method for measuring in vivo the glucose content in blood.

The present invention is particularly suited for application with devices and methods of the type described in WO2002/069791, WO2005/053526 or WO2005/053523. Apart from a measurement of glucose, the invention can also be advantageously used in other types of measurements. In particular, it can be used for any type of measurements that measure the electrical impedance of the tissue, such as humidity measurements. As mentioned, it can also be used for optical measurements, such as glucose measurements or heart pulse measurements based at least in part on measurements of optical absorption or reflection.

The application of the present invention for the measurement of glucose in particularly advantageous because the accuracy of conventional optical or electrical measurements suffers degradation caused by an excessive sweat generation when the body is in a hypoglycemic state. Thus, the invention relates to a method for measuring in vivo the glucose content in blood in the hypoclycemic and non-hypoglcemic state. The method according to this invention is particular suitable, as it is believed that mnAChR antagonist prevents or reduces such sweat generation in the treated skin area.

Advantageously, the mnAChR antagonist as described herein is applied transdermally.

The examples below are provided to further illustrate the invention. These examples are not intended to limit the invention as described herein.

### EXAMPLE 1

### Manufacture of a Topical Composition: Cream

1.1 General procedure: First all lipophilic substances (no.1-3) and all hydrophilic substances (no4, 5) are combined, mixed and heated to 60°-80° C. separately to obtain two homogeneous phases. Afterwards the water (hydrophilic) phase is poured under stirring into the lipophilic phase to obtain an emulsion. The emulsion is cooled down to about 35° C., additional substances as ethereal oils, active ingredients (including mnAChR antagonist) and preservatives (no. 6-8) are added by stirring into the emulsion. A basic formula for an o/w cream is given below:

| no. | Ingredient | Phase | % w/w |
|---|---|---|---|
| 1 | Plant oil/mineral oil | A | 30 |
| 2 | Emulsifiers (e.g. sugrose | lipophilic | |
| 3 | stearate) | | |
| | Water phase thickener (e.g. | | |
| | xanthan gum) | | |
| 4 | Water | B | 70 |
| 5 | Water soluble, heat resistant | hydrophilic | |
| | substances | | |
| 6 | Ethereal oils | | |
| 7 | Preservatives (e.g. parabens) | | |
| 8 | Active ingredients (e.g. urea) | | |

1.2 W/o cream, suitable for dry skin: A soft cream is obtained by melting components A-D in a water bath at 80° C. Component E is melted separately in a water bath and then poured into the mixture of components A-D. Component F is dissolved in G and heated to 90° C. Components H, I, K and L are added to the aqueous phase (F+G). Finally, the aqueous phase is added to the lipophilic phase and emulsified. The cream obtained is filled into an aluminum tube and stored in the fridge at 2-8° C.

| Ingredients | Amount | |
|---|---|---|
| A Paraffinum liquidum | 32.5 | g |
| B Petrolatum | 22.7 | g |
| C Glycerol monostearate 40-50 | 3.2 | g |
| D Sorbitan monostearate | 2.1 | g |
| E Polyoxyethylenglykol(30)-stearate | 4.4 | g |
| F citric acid, monohydrate | 0.1 | g |
| G water, puriss. | 21.0 | g |
| H urea | 5.0 | g |
| I Glycerol 85% | 8.5 | g |
| K Phenoxetol | 0.5 | g |
| L compound of formula (I) 0.25% in | 10 | mg |
| water/glycerin 70/30 | | |

1.3 o/w cream: Components A and B are melt at 55-60° C., than components C and D are added. Component E is dissolved in 7.5 ml F (solution 1). The remaining part of F is added and shaken for 5 min to homogenise the phases. All phases are slowly cooled down to 25° C., afterwards solution 1 is add, than the water is added and homogenized. Finally, component F is added and further homogenized.

| | |
|---|---|
| Ingredients | Amount |

| | | |
|---|---|---|
| A Cetyl alcohol | 21.0 | g |
| B Paraffinum liquidum | 19.0 | g |
| C Sorbitan monooleate 80 | 0.5 | g |
| D Polysorbat 80 | 4.5 | g |
| E Chlorhexidinglucoante solution 20% | 0.5 | g |
| F water puriss. | 54.5 | g |
| G compound of formula (I) 0.25% in | 10 mg | |
| water/glycerin 70/30 | | |

1.4 o/w cream: Components A-H are melt and homogenized at 65° C. Components J, K, L, M and N are added to I, and dissolved under stirring and heating to 70° C. The thus obtained water phase is added under stirring to the above prepared lipophilic phase. The obtained emulsion is cooled to 35° C. and components N, O, P are added.

| | Amount |
|---|---|
| Ingredients | [%] |
| A Lanolin alcohol | 2.4 |
| B Cetearyl alcohol | 1.5 |
| C Paraffinum Liquidum | 7.5 |
| D Oenothera biennis | 8.0 |
| E Petrolatum | 1.7 |
| F Paraffinum (Paraffinwachs) | 2.1 |
| G Prunus dulcis | 3.0 |
| H Ozokerite | 3.0 |
| I water puriss | 50.8 |
| J Urea | 5.0 |
| K Lactic acid | 5.0 |
| L Hyaluronic acid, sodium salt | 0.01-0.1 |
| M Glycerol | 2.8 |
| N Magnesium Sulfate | 1.0 |
| O Dexpanthenole | 2.0 |
| P preservative | qs |
| Q compound of formula (I); (solution, 3% in | 4.0 |
| water/glycerin 70/30) | |

1.5 gel: Components A-H are mixed at 50° C., than component I is added slowly, the obtained gel-like composition is cooled down to r.t. and component J is added

| | Amount |
|---|---|
| Ingredients | [%] |
| A citric acid, water free, Ph. Eur. | |
| B 1,2 propylenglycole, Ph. Eur. | |
| C sodium edetate | |
| D ethanol puriss, 96 Vol. % | |
| E methyl-4-hydroxybenzoate, Ph. Eur. (Nipagin) | |
| F propyl-4-hydroxybenzoat, Ph. Eur. (Nipasol) | |
| G acryli acid polymerisate 934 PH | |
| H water puriss Ph. Eur. | |
| I NaOH 10% | |
| J compound of formula (I); (solution, 3% in | |
| water/glycerin 70/30) | |

1.6 cream: Components F-J are melt and homogenized at 65° C. Components A-D are added to E, and dissolved under stirring and heating to 70° C. The thus obtained water phase is added under stirring to the above prepared lipophilic phase. The obtained emulsion is cooled to 35° C. and component K is added.

| | Amount |
|---|---|
| Ingredients | [%] |
| A citric acid, water free, Ph. Eur. | |
| B 1,2 propylenglycole, Ph. Eur. | |
| C methyl-4-hydroxybenzoate, Ph. Eur. (Nipagin) | |
| D propyl-4-hydroxybenzoat, Ph. Eur. (Nipasol) | |
| E water puriss Ph. Eur. | |
| F Paraffinum | |
| G Cetylalkohol, Ph. Eur. | |
| H Stearylalkohol, Ph. Eur. | |
| I Polysorbat 60, Ph. Eur. (Tween 60) | |
| J Sorbitanmonostearat, Ph. Eur. (Span 60) | |
| K compound of formula (I); (solution, 3% in | |
| water/glycerin 70/30) | |

### EXAMPLE 2

### Measuring of Impedance Spectrum Using a Topical Composition According to Ex. 1

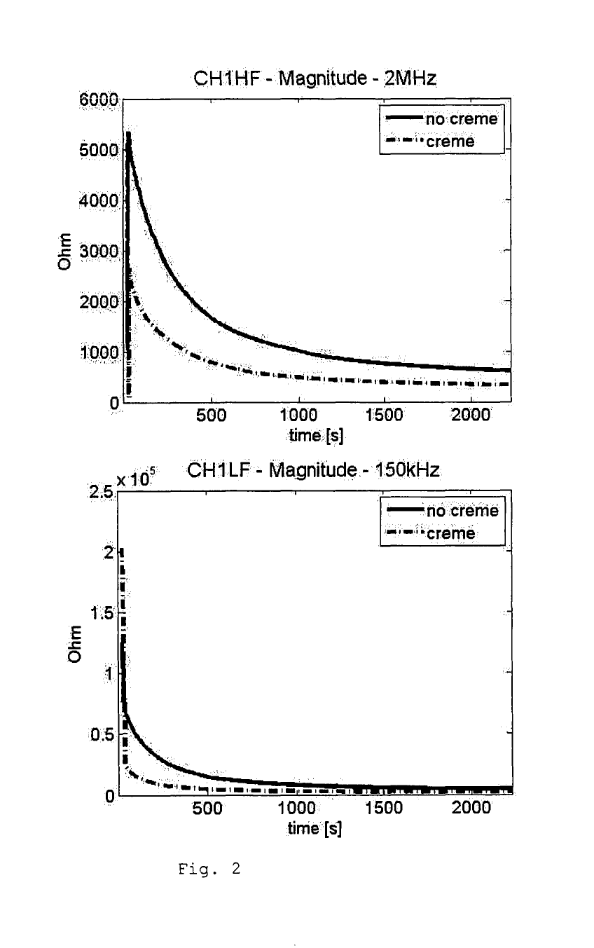

The impedance is measured with a differential sensor featuring two electrodes, 200 um and 4 mm wide. The electrodes are driven by a hardware, allowing to measure magnitude and phase at various frequencies. Such devices are known and disclosed e.g. in WO2005/120332, WO2007/053963. Results are shown in FIG. 2 at 2 MHz (above) and 150 kHz (below), wherein the dotted line refers to a measurement with a composition as described herein, while the straight line refers to an untreated comparative measurement. These results clearly prove the stabilizing effect of a composition as described herein.

1-15. (canceled)

16. Method for measuring in vivo the glucose content in blood, comprising the steps of:
i) treating a skin area of a body with a topical composition comprising one or more reversible mnAChR-antagonists,
ii) applying to said skin area a measuring device,
iii) measuring by means of said measuring device at least one parameter depending on a response to an electromagnetic field and
iv) determining the glucose-content.

17. Method according to claim 16,
a. wherein an electromagnetic field is applied by means of one or more field generating electrodes, and
b. wherein said measuring device comprises one or more measuring electrodes.

18. A method according to claim 16 where said electromagnetic field in is an AC field having a frequency in the range of 1 kHz to 10 GHz.

19. Method according to claim 16,
a. wherein an electromagnetic field is applied by means of a light source emitting UV-, visible or infrared light, and
b. wherein said measuring device comprises one or more light sensitive sensors.

20. A method according to claim 19 wherein said electromagnetic field is light of a wave-length between 0.2-100 μm.

21. A method according to claim 20 wherein said light is of a wavelength larger than 0.8 μm.

22. Method according to claim 16, wherein said mnAChR-antagonist is selected from the group consisting of natural and synthetic tripeptides.

23. Method according to claim 22 wherein said tripeptide is a compound of formula (I) 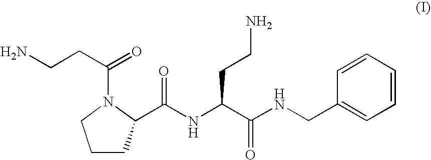
or salt thereof.

24. A method according to claim 23, wherein said compound is the diacetate salt.

25. Method according to claim 16, wherein said topical composition is a liquid, gel or cream.

26. Topical composition, comprising
a. 0.1 to 0.001 wt-% of one or more mnAChR-antagonist;
b. 0.01-10 wt-% wt-% hyaluronic acid; and
c. optionally one or more occlusive agents; option-ally one or more humectants; optionally one or more emollients.

27. Topical composition according to claim 26, wherein said mnAChR-antagonist, is selected from the group consisting of natural and synthetic tripeptides.

28. Topical composition according to claim 26, additionally comprising up to 30 wt-% glycerol and optionally up to 10 wt-% urea.

29. Topical composition according to claim 26, in the form of a solution, gel or cream.

30. A single sachet containing two compartments, wherein one compartment contains a topical composition according to claim 26, the other compartment contains a cleansing composition.

31. The method according to claim 16, wherein the topical composition employed comprises:
a. 0.1 to 0.001 wt-% of one or more mnAChR-antagonist;
b. 0.01-10 wt-% wt-% hyaluronic acid;
c. optionally one or more occlusive agents; optionally one or more humectants; optionally one or more emollients.

32. The method according to claim 31, wherein said mnAChR-antagonist from the group consisting of natural and synthetic tripeptides.
